(19) United States
(12) Patent Application Publication
(10) Pub. No.: US 2025/0262025 A1
(43) Pub. Date: Aug. 21, 2025

(54) ANTIBACTERIAL COATING FOR DENTAL CROWN AND BRIDGE FITTING SURFACE TO REDUCE SECONDARY CARIES

(71) Applicant: KING FAISAL UNIVERSITY, Al-Ahsa (SA)

(72) Inventors: ZOHAIB KHURSHID, AL AHSA (SA); Muhammad FAHEEMUDDIN, AL-AHSA (SA); BINOY MATHEWS NEDUMGOTTIL, AL-AHSA (SA); RAMY MOUSTAFA ALI, AL-AHSA (SA)

(21) Appl. No.: 18/444,521

(22) Filed: Feb. 16, 2024

(51) Int. Cl. A61C 3/025; A61K 6/17; A61K 6/824; A61K 6/831

(52) U.S. Cl. CPC A61C 3/025; A61K 6/17; A61K 6/824; A61K 6/831

(57) ABSTRACT

A dental crown having antibacterial nanoparticles embedded thereon and a method of obtaining the same. The dental crown is prepared by sandblasting the crown with the antibacterial nanoparticles to increase the surface porosity and roughness of an intaglio surface of the dental crown. This procedure can be used to enhance metal and/or zirconia crown fitting surfaces for improving bonding and increasing clinical durability. During this procedure, the antibacterial nanoparticles which are biocompatible to the human body will further be introduced to reduce secondary caries and help to provide protection of the prepared tooth. The antibacterial nanoparticles can be graphene oxide (GEO).

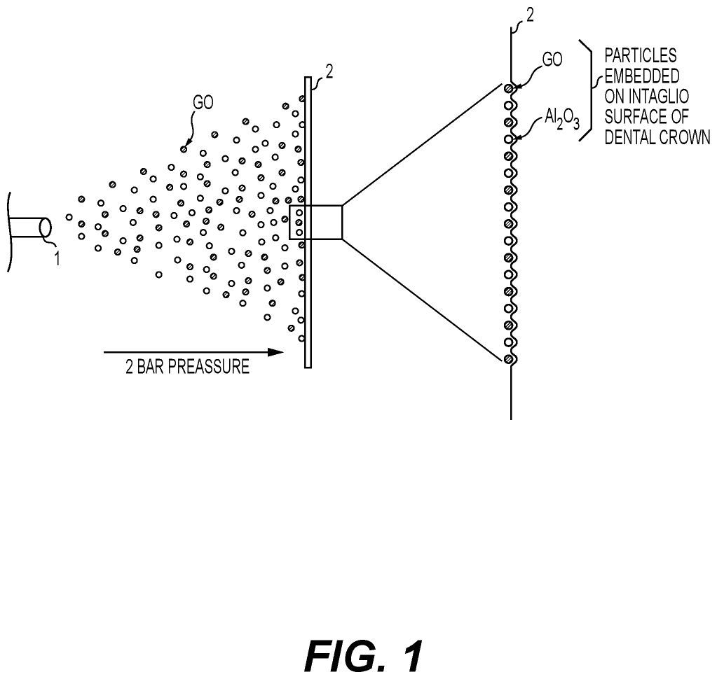

### BACKGROUND

### 1. Field

The disclosure of the present patent application relates to a dental crown having sprayed antibacterial nanoparticles, a method of obtaining the same, and a method of reducing secondary caries using the same.

### 2. Description of the Related Art

Prosthetic devices such as inlays, onlays, veneers, and crowns are used to replace carious or discolored tooth structure and restore the normal function of defective tooth structure. Although quite durable and easy to cement, prosthetic devices made of metallic material such as gold alloy, stainless steels, and base-metal alloys (e.g. nickel-chromium alloys) are optically opaque and non-aesthetic.

Over the past two decades, tooth colored and aesthetic prosthetic devices made of ceramic materials, indirect composite resins, and metal oxide materials have increasingly become the choice of restorative materials for both dental patients and practitioners due to their unmatched aesthetics. Examples of ceramic materials include porcelain, feldspathic porcelain, aluminous porcelain, leucite reinforced ceramic material, lithium disilicate reinforced ceramic material, glass-infiltrated magnesia aluminate spinell, glass-infiltrated alumina, glass-infiltrated zirconia, and alumina. Examples of metal oxide materials include alumina, zirconia, and yttrium stabilized zirconia. There are numerous antibacterial materials incorporated in dental composites and denture acrylic materials previously reported such as graphene, titania nanoparticles, silver, and copper.

The non-opaque and tooth colored restoration requires use of a resin cement to adhesively bond it to tooth structure because of resin cement's excellent aesthetic qualities (color matching ability and good translucency). However, current resin cements are hydrophobic and have no self-adhesive properties, and therefore require a more complex cementation procedure including etching, priming/bonding, and cementing steps.

A typical cementation procedure for cementing a ceramic prosthetic device to tooth structure is as follows. If the patient wears a temporary prosthetic device, the temporary prosthetic device along with the temporary cement will be removed first. The tooth will be thoroughly cleaned typically using pumice cleaning and subsequently rinsed with water. The cementation process involves following steps: (1) the tooth is first etched with an acidic etchant to remove the smear layer on the tooth surface and also to create a more retentive surface for bonding; (2) a dental primer (for some adhesive systems) is applied to the tooth surface; (3) a dental adhesive is then applied to the tooth surface; (4) the dental adhesive is light-cured; (5) the ceramic bonding surface is air-abraded with aluminum oxide particles (optional); (6) the ceramic surface is then etched with hydrofluoric acid; (7) the ceramic surface is coated with a silane primer; (8) the ceramic surface is then coated with a primer/adhesive (for most cementation system); (9) the ceramic surface is bonded to the tooth with a light-curable or dual-curable (light-curable and self-curable) resin cement; and finally (10) the resin cement is hardened by light-curing or dual-curing (light-curing and self-curing). The current procedures for cementing a dental ceramic veneer restoration to tooth structure is rather cumbersome, involves many steps, and therefore is quite technique sensitive and time consuming.

It is highly desirable to simplify the above cementation procedures, shorten the chair time for the dental practitioner, increase bond strength and increase the reduction of secondary dental caries.

### SUMMARY

The present subject matter is directed to a dental crown having antibacterial nanoparticles sprayed thereon and a method of obtaining the same.

In this regard, the present subject matter relates generally to a new method of sandblasting used to increase the surface porosity and roughness of metal surfaces of dental crowns. This procedure can be used to enhance metal and/or zirconia crown fitting surfaces for improving bonding and increasing clinical durability. During this procedure, antibacterial nanoparticles which are biocompatible to the human body will further be introduced to reduce the secondary caries and help to provide protection of the prepared tooth (either prepared for crown or bridge).

The present subject matter relates to dental crowns having graphene oxide (GEO) antibacterial nanoparticles incorporated therewith. In certain embodiments, these dental crowns can be prepared by sandblasting, with the sandblasting being performed with a sandblasting machine. The sandblasting can help to enhance the bonding of dental luting cement and improve the overall esthetics of the crown.

Accordingly, in an embodiment, the present subject matter relates to a method for reducing secondary caries in a mouth of a patient to which a dental crown is applied, the method comprising: sandblasting the dental crown with antibacterial nanoparticles to increase surface porosity and roughness of an intaglio surface of the dental crown and sprayed the antibacterial nanoparticles on the intaglio surface of the dental crown; and applying the dental crown to a tooth of the patient prepared to accept the dental crown.

In another embodiment, the present subject matter relates to a dental crown configured to reduce secondary caries in a mouth of a patient to which the dental crown is applied, the dental crown comprising: a dental crown made of nickel chromium, zirconia, or a combination thereof having antibacterial nanoparticles embedded thereon to increase surface porosity and roughness of an intaglio surface of the dental crown.

These and other features of the present subject matter will become readily apparent upon further review of the following specification.

### BRIEF DESCRIPTION OF THE DRAWINGS

The sole FIGURE pictorially demonstrates the present sandblasting procedure.

### DETAILED DESCRIPTION OF THE PREFERRED EMBODIMENTS

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

The present subject matter is directed to a dental crown having antibacterial nanoparticles embedded thereon and a method of obtaining the same.

In this regard, the present subject matter relates generally to a new method of sandblasting used to increase the surface porosity and roughness of metal surfaces of dental crowns. This procedure can be used to enhance metal and/or zirconia crown fitting surfaces for improving bonding and increasing clinical durability. During this procedure, antibacterial nanoparticles which are biocompatible to the human body will further be introduced to reduce the secondary caries and help to provide protection of the prepared tooth (either prepared for crown or bridge).

The present subject matter relates to dental crowns having graphene oxide (GEO) antibacterial nanoparticles incorporated therewith. In certain embodiments, these dental crowns can be prepared by sandblasting, with the sandblasting being performed with a sandblasting machine. The sandblasting can help to enhance the bonding of dental luting cement and improve the overall esthetics of the crown.

Accordingly, in an embodiment, the present subject matter relates to a method for reducing secondary caries in a mouth of a patient to which a dental crown is applied, the method comprising: sandblasting the dental crown with antibacterial nanoparticles to increase surface porosity and roughness of an intaglio surface of the dental crown and embed the antibacterial nanoparticles on the intaglio surface of the dental crown; and applying the dental crown to a tooth of the patient prepared to accept the dental crown.

In one embodiment, the dental crown can be made of nickel chromium, zirconia, or a combination thereof.

In another embodiment, the antibacterial nanoparticles can comprise graphene oxide nanoparticles. In an alternative embodiment, the antibacterial nanoparticles can comprise graphene oxide nanoparticles and aluminum oxide (Al₂O₃) nanoparticles. In this regard, the graphene oxide and aluminum oxide nanoparticles can have an average particle size of about 40 to about 60 microns, or about 50 microns.

In an additional embodiment, the sandblasting can improve bonding of dental luting cement used with the dental crown and durability of the dental crown. As a result, the dental crown can protect the tooth of the patient prepared to accept the dental crown.

In certain embodiments, the sandblasting can be performed with a sandblasting machine. In this regard, the sandblasting can be conducted using an air pressure of about 40 psi to about 60 psi. In one non-limiting example, and as seen in the sole figure, the sandblasting machine can comprise a sandblasting device tip performing the sandblasting at about 2 bars of pressure for about 10 seconds at a distance of about 10 mm from the dental crown, thereby embedding the intaglio surface of the dental crown with the antibacterial nanoparticles described herein.

In another embodiment, the present subject matter relates to a dental crown configured to reduce secondary caries in a mouth of a patient to which the dental crown is applied, the dental crown comprising: a dental crown made of nickel chromium, zirconia, or a combination thereof having antibacterial nanoparticles embedded thereon to increase surface porosity and roughness of an intaglio surface of the dental crown.

In this regard, the antibacterial nanoparticles are biocompatible to the human body and can be embedded on the dental crown, i.e., on the intaglio surface of the dental crown via sandblasting, thereby increasing surface porosity and roughness of the intanglio surface of the dental crown. Further in this regard, the antibacterial nanoparticles can comprise graphene oxide nanoparticles, or graphene oxide nanoparticles and aluminum oxide (Al₂O₃) nanoparticles. In certain embodiments, the graphene oxide and aluminum oxide nanoparticles can have an average particle size of about 40 to about 60 microns, or about 50 microns. The presence of the nanoparticles can reduce the secondary caries in the mouth of a patient to which the present dental crown is applied and help to provide protection of the prepared tooth (either prepared for a crown or a bridge).

It is to be understood that the dental crown is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A method for reducing secondary caries in a mouth of a patient to which a dental crown is applied, the method comprising:
sandblasting the dental crown with antibacterial nanoparticles to increase surface porosity and roughness of an intaglio surface of the dental crown and embed the antibacterial nanoparticles on the intaglio surface of the dental crown; and
applying the dental crown to a tooth of the patient prepared to accept the dental crown.

2. The method of claim 1, wherein the dental crown is made of nickel chromium, zirconia, or a combination thereof.

3. The method of claim 1, wherein the antibacterial nanoparticles comprise graphene oxide nanoparticles.

4. The method of claim 1, wherein the antibacterial nanoparticles comprise graphene oxide nanoparticles and aluminum oxide (Al₂O₃) nanoparticles.

5. The method of claim 4, wherein the graphene oxide and aluminum oxide nanoparticles have an average particle size of about 40 to about 60 microns.

6. The method of claim 1, wherein the sandblasting improves bonding of dental luting cement used with the dental crown and durability of the dental crown.

7. The method of claim 1, wherein the dental crown protects the tooth of the patient prepared to accept the dental crown.

8. The method of claim 1, wherein the sandblasting is performed with a sandblasting machine.

9. The method of claim 1, wherein the sandblasting is conducted using an air pressure of about 40 psi to about 60 psi.

10. The method of claim 8, wherein the sandblasting machine comprises a sandblasting device tip performing the sandblasting at about 2 bars of pressure for about 10seconds at about 10 mm from the dental crown.

11. A dental crown configured to reduce secondary caries in a mouth of a patient to which the dental crown is applied, the dental crown comprising:
a dental crown made of nickel chromium, zirconia, or a combination thereof having antibacterial nanoparticles embedded thereon to increase surface porosity and roughness of an intaglio surface of the dental crown.

12. The dental crown of claim 11, wherein the antibacterial nanoparticles are embedded on the dental crown via sandblasting, thereby increasing surface porosity and roughness of the intaglio surface of the dental crown.

13. The dental crown of claim 11, wherein the antibacterial nanoparticles comprise graphene oxide nanoparticles.

14. The dental crown of claim 13, wherein the antibacterial nanoparticles comprise graphene oxide nanoparticles.

15. The dental crown of claim 14, wherein the graphene oxide and aluminum oxide nanoparticles have an average particle size of about 40 to about 60 microns.
